(19) United States
(12) Patent Application Publication
(10) Pub. No.: US 2025/0288812 A1
(43) Pub. Date: Sep. 18, 2025

(54) IMPROVED PROGRAMMING OF NEURAL STIMULATION THERAPY

(71) Applicant: Saluda Medical Pty Limited, Macquarie Park, New South Wales (AU)

(72) Inventors: Milan Obradovic, Artarmon (AU); Dean Michael Karantonis, Artarmon (AU); Daniel John Parker, Artarmon (AU)

(21) Appl. No.: 18/859,726

(22) PCT Filed: Apr. 28, 2023
(86) PCT No.: PCT/AU2023/050356

§ 371 Date: Oct. 24, 2024

(30) Foreign Application Priority Data
Apr. 28, 2022 (AU) 2022901117

(51) Int. Cl. A61N 1/36; A61N 1/02

(52) U.S. Cl. CPC A61N 1/36139; A61N 1/025; A61N 1/36125

(57) ABSTRACT

A neural stimulation system comprising: a neuromodulation device for delivering neural stimuli, and a processor. The neurostimulation device comprises: a plurality of electrodes; a stimulus source to provide neural stimuli to be delivered via the electrodes to a neural tissue to evoke neural responses; measurement circuitry configured to capture signal windows sensed at the electrodes subsequent to respective neural stimuli; and a control unit configured to control the stimulus source to provide each neural stimulus according to a stimulus intensity parameter. The processor is configured to: instruct the control unit to control the stimulus source to sequentially provide a plurality of neural stimuli according to a ramp of stimulus intensity parameter values up to a perceptual marker; receive a captured signal window subsequent to each neural stimulus; detect whether an evoked neural response is present in each signal window; and determine the perceptual marker based on the detecting.

The present application claims priority from Australian Provisional Patent Application No 2022901117 filed on 28 Apr. 2022, the contents of which are incorporated herein by reference in their entirety.

### TECHNICAL FIELD

The present invention relates to neural stimulation therapy and in particular to determining key parameters of a patient's response to stimuli during such therapy.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Action potentials propagating along Aβ (A-beta) fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Another control problem facing neuromodulation devices of all types is achieving neural recruitment at a sufficient level for therapeutic effect, but at minimal expenditure of energy. The power consumption of the stimulation paradigm has a direct effect on battery requirements which in turn affects the device's physical size and lifetime. For rechargeable devices, increased power consumption results in more frequent charging and, given that batteries only permit a limited number of charging cycles, ultimately this reduces the implanted lifetime of the device.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

Closed-loop neural stimulation therapy is governed by a number of parameters to which values must be assigned to implement the therapy. The effectiveness of the therapy depends in large measure on the suitability of the assigned parameter values to the patient undergoing the therapy. As patients vary significantly in their physiological characteristics, a “one-size-fits-all” approach to parameter value assignment is likely to result in ineffective therapy for a large proportion of patients. An important preliminary task, once a neuromodulation device has been implanted in a patient, is therefore to assign values to the therapy parameters that maximise the effectiveness of the therapy the device will deliver to that particular patient. This task is known as programming or fitting the device. Programming generally involves applying certain test stimuli via the device, recording responses, and based on the recorded responses, inferring or calculating the most effective parameter values for the patient. The resulting parameter values are then formed into a “program” that may be loaded to the device to govern subsequent therapy. Some of the recorded responses may be neural responses evoked by the test stimuli, which provide an objective source of information that may be analysed along with subjective responses elicited from the patient. In an effective programming system, the more responses that are analysed, the more effective the eventual assigned parameter values should be.

However, programming may be costly and time-consuming if unnecessarily prolonged. There is therefore an incentive to minimise the number of test stimuli to be applied and the amount of information to be recorded and analysed in order to produce the assigned values of the therapy parameters. Moreover, thresholds for discomfort vary widely between patients, between postures for a single patient, and between stimulus electrodes for a given patient in a given posture. It is difficult to know in advance where a given patient's discomfort threshold is in a given posture. The result is that a test stimulus of an intensity that is comfortable for one patient may provoke acute discomfort for another patient, or for the same patient in a different posture, or for the same patient in the same posture when applied at a different stimulus electrode. This complicates certain aspects of programming involving measurement of the intensity of patients' neural responses across the full comfortable range of stimulus intensity at a particular stimulus electrode in a particular posture.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are methods and systems for rapidly estimating key parameters of a patient's activation plot without causing discomfort. The disclosed methods and systems use measurements of neural response intensity, which are a more reliable and consistent measure of patient sensation than stimulus intensity, to estimate the key parameters without requiring any patient reporting of their sensations. The stimulus intensity is ramped up from zero until a neural response is consistently detected, and the stimulus intensity at that point is marked as the provisional ECAP threshold. The stimulus intensity ramp continues to a perceptual marker, determined dynamically from the provisional ECAP threshold, recording response intensities for each stimulus. Activation plot models are fitted to the measured response intensities above and below the provisional ECAP threshold. From the fitted models, the key parameters of the activation plot may be obtained.

According to a first aspect of the present technology, there is provided a neural stimulation system comprising: a neuromodulation device for delivering neural stimuli, and a processor. The neuromodulation device comprises: a plurality of implantable electrodes including one or more stimulus electrodes and one or more sense electrodes; a stimulus source configured to provide neural stimuli to be delivered via the one or more stimulus electrodes to neural tissue of a patient in order to evoke neural responses in the neural tissue; measurement circuitry configured to capture signal windows sensed at one or more sense electrodes subsequent to respective neural stimuli; and a control unit configured to control the stimulus source to provide each neural stimulus according to a stimulus intensity parameter. The processor is configured to: instruct the control unit to control the stimulus source to sequentially provide a plurality of neural stimuli according to a ramp of stimulus intensity parameter values up to a perceptual marker; receive a captured signal window subsequent to each neural stimulus; detect whether an evoked neural response is present in each captured signal window; and determine the perceptual marker based on the detecting.

According to a second aspect of the present technology, there is provided an automated method of delivering neural stimuli to a neural tissue of a patient. The method comprises: sequentially delivering a plurality of neural stimuli to the neural tissue according to a ramp of stimulus intensity parameter values up to a perceptual marker; receiving a captured signal window subsequent to each delivered neural stimulus; detecting whether an evoked neural response is present in each captured signal window; and determining the perceptual marker based on the detecting.

According to a third aspect of the present technology, there is provided a neural stimulation system comprising: a neuromodulation device for delivering neural stimuli, the neuromodulation device comprising: a plurality of implantable electrodes including one or more stimulus electrodes and one or more sense electrodes; a stimulus source configured to provide neural stimuli to be delivered via the one or more stimulus electrodes to neural tissue of a patient in order to evoke neural responses in the neural tissue; measurement circuitry configured to capture signal windows sensed at one or more sense electrodes subsequent to respective neural stimuli; and a control unit configured to control the stimulus source to provide each neural stimulus according to a stimulus intensity parameter; and a processor configured to: instruct the control unit to control the stimulus source to provide one or more neural stimuli according to a stimulus intensity parameter value; detect whether an evoked neural response is present in each captured signal window; increment the stimulus intensity parameter value; repeat the instructing, detecting and incrementing until the stimulus intensity parameter value is equal to a perceptual marker; and determine the perceptual marker based on the detecting.

According to a fourth aspect of the present technology, there is provided an automated method of delivering neural stimuli to neural tissue of a patient, the method comprising: delivering one or more neural stimuli to the neural tissue according to a stimulus intensity parameter value; detecting whether an evoked neural response is present in each captured signal window; incrementing the stimulus intensity parameter value; repeating the delivering, detecting and incrementing until the stimulus intensity parameter value is equal to a perceptual marker; and determining the perceptual marker based on the detecting.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIGS. 8A and 8B are a flow chart illustrating a method of estimating key parameters of the patient's response to stimulus, according to one aspect of the present technology;

FIG. 9 is a graph 900 illustrating the operation of the method of FIG. 8; and

FIG. 10 is a flow chart 1000 illustrating a method in accordance to one implementation of the present technology.

### DETAILED DESCRIPTION OF THE PRESENT TECHNOLOGY

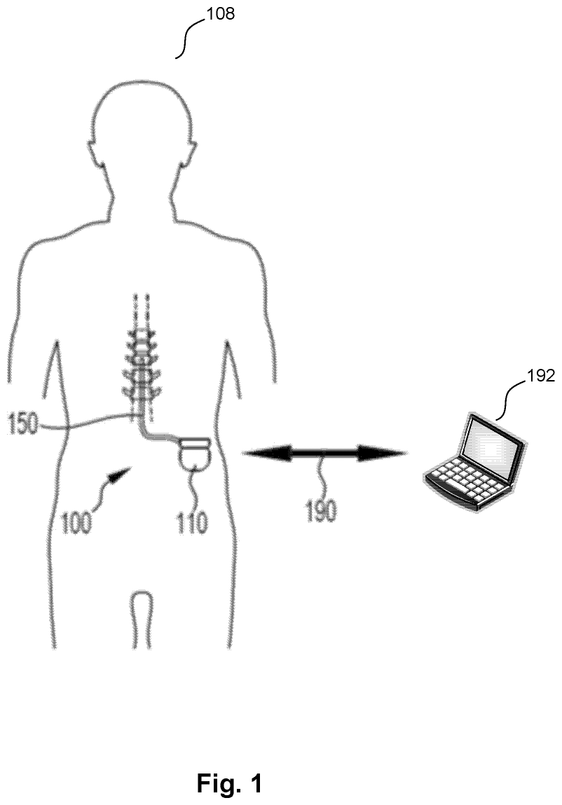

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

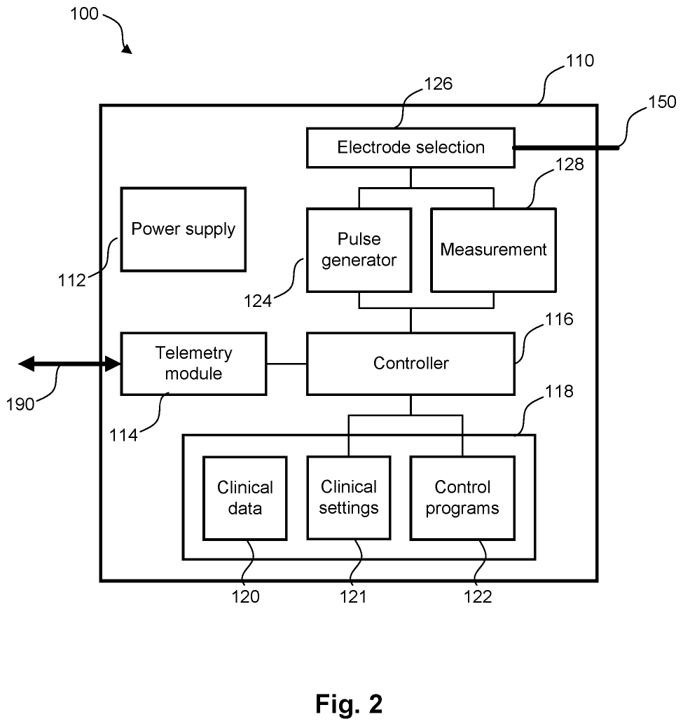

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 controls a pulse generator 124 to generate stimuli, such as in the form of electrical pulses, in accordance with the clinical settings 121 and control programs 122. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

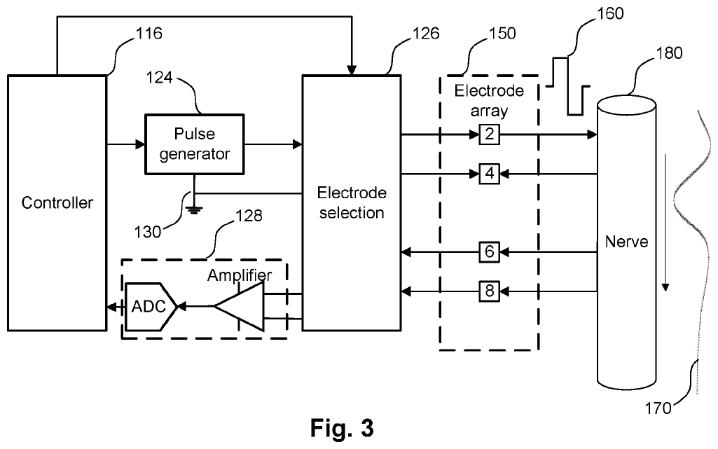

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes. The set of stimulus and return electrodes and their respective polarities is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

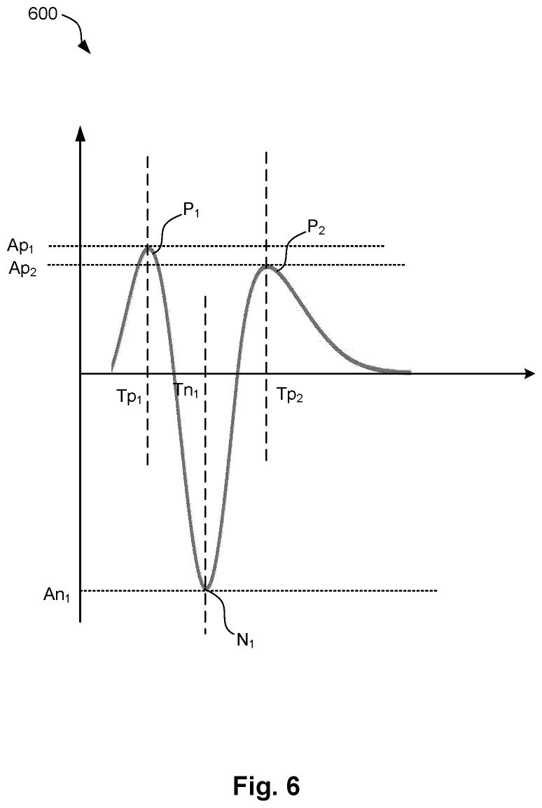

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak PI is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183 by the present applicant.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

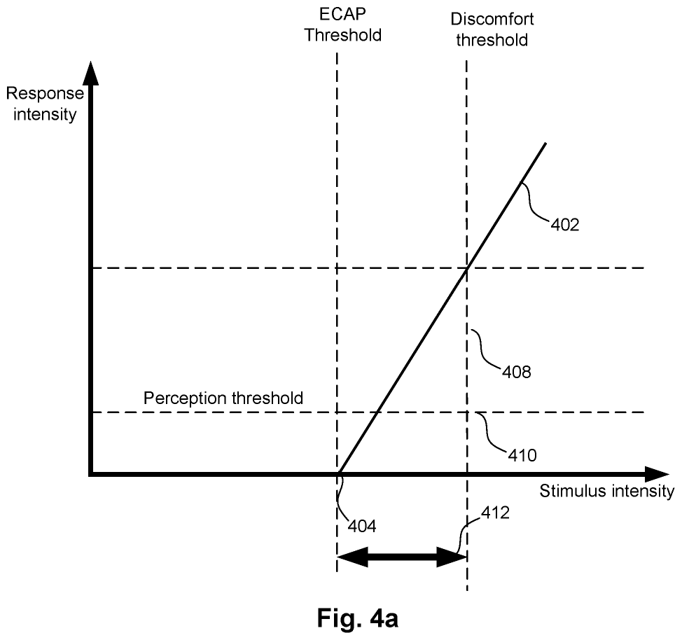

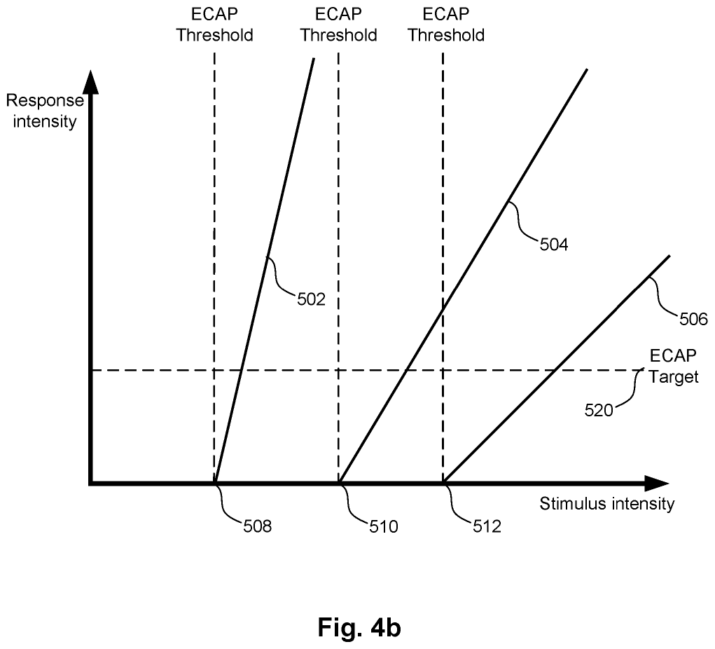

An activation plot, or growth curve, is an approximation to the relationship between stimulus intensity (e.g. an amplitude of the current pulse 160) and intensity of neural response 170 resulting from the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s \geq T\end{matrix} \qquad ( 1 )$$

where s is the stimulus intensity, y is the ECAP amplitude, T is the ECAP threshold and S is the slope of the activation plot (referred to herein as the patient sensitivity). The slope S and the ECAP threshold T are the key parameters of the activation plot 402.

FIG. 4a also illustrates a discomfort threshold 408 (sometimes referred to as a comfort threshold), which is a stimulus intensity above which the patient 108 experiences uncomfortable or painful stimulation. FIG. 4a also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is detected, and its amplitude measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

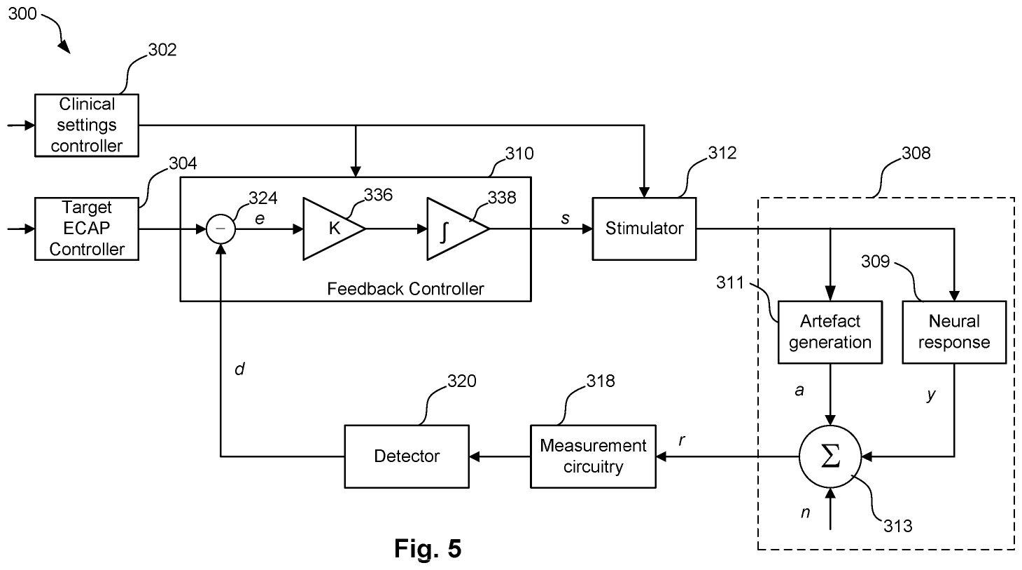

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including electrical noise from external sources such as 50 Hz mains power; electrical disturbances produced by the body such as neural responses evoked not by the device but by other causes such as peripheral sensory input, EEG, EMG, and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and measurement noise) and samples the amplified sensed signal r to capture a “signal window” comprising a predetermined number of samples of the amplified sensed signal r. The ECAP detector 320 processes the signal window and outputs a measured neural response intensity d. A typical number of samples in a captured signal window is 60. In one implementation, the neural response intensity comprises an ECAP amplitude. The measured response intensity d is input into the feedback controller 310. The feedback controller 310 comprises a comparator 324 that compares the measured response intensity d to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameters to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be computed by the feedback controller 310 as

$$s = \int K e d t \qquad ( 2 )$$

where K is the gain of the gain element 336 (the controller gain). This relation may also be represented as

δs=Ke
where δs is an adjustment to the current stimulus intensity parameter s.

A target ECAP amplitude is input to the comparator 324 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the CLNS system 300, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the gain K for the gain element 336 and the stimulus parameters for the stimulator 312. The clinical settings controller 302 may be configured to adjust the gain K of the gain element 336 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

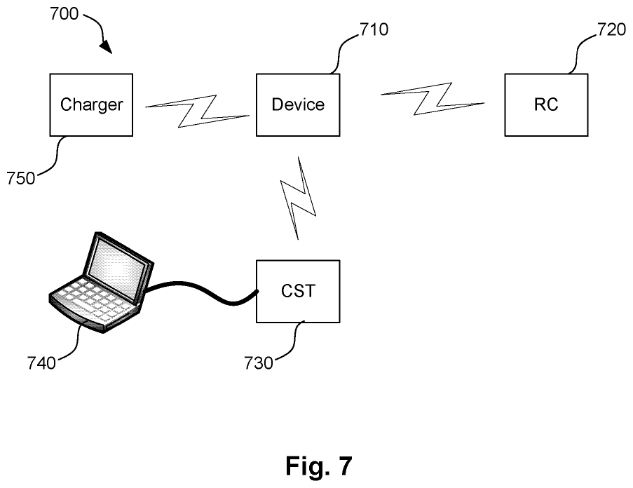

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### The Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. There is therefore a need for a CPA to be as intuitive for non-technical users as possible while avoiding discomfort to the patient.

Implementations of an Assisted Programming System (APS) according to the present technology are generally configured to meet this need. In some implementations, the APS comprises two elements: the Assisted Programming Module (APM), which forms part of the CPA, and the Assisted Programming Firmware (APF), which forms part of the control programs 122 executed by the controller 116 of the electronics module 110. The data obtained from the patient is analysed by the APM to determine the parameters and settings for the neural stimulation therapy to be delivered by the stimulator 100. The APF is configured to complement the operation of the APM by responding to commands issued by the APM via the CST 730 to the stimulator 100 to deliver specified stimuli to the patient, and by returning, via the CST 730, measurements of neural responses to the delivered stimuli.

In other implementations, all the processing of the APS according to the present technology is done by the APF. In other words, the data obtained from the patient is not passed to the APM, but is analysed by the APF to determine the parameters and settings for the neural stimulation therapy to be delivered by the stimulator 100.

In implementations of the APS in which the APM analyses the data from the patient, the APS instructs the device 710 to capture and return signal windows to the CI 740 via the CST 730. In such implementations, the device 710 captures the signal windows using the measurement circuit 128 and bypasses the ECAP detector 320, storing the data representing the raw signal windows temporarily in memory 118 before transmitting the data representing the captured signal windows to the APS for analysis.

Following the processing, the APS may load the determined program onto the device 710 to govern subsequent neural stimulation therapy. In one implementation, the program comprises clinical settings 121, also referred to as therapy parameters, that are input to the neuromodulation device by, or stored in, the clinical settings controller 302. The patient may subsequently control the device 710 to deliver the therapy according to the determined program using the remote controller 720 as described above. The determined program may also, or alternatively, be loaded into the CPA for validation and modification.

### Noise Departure Detector (NDD)

In some implementations, the APS according to the present technology comprises a noise departure detector (NDD). The NDD is a statistical detector of the presence of an ECAP in a signal window. The operation of the NDD on a signal window is preferably preceded by an “artefact scrubber” which removes artefact from the signal window. On such artefact scrubber is disclosed by the present applicant in International Patent Publication no. WO2020/124135, the entire contents of which are herein incorporated by reference.

The NDD works by detecting a statistically unusual difference from the expected noise present in a signal window. The extent of the difference is indicative of the likelihood of an ECAP in the signal window.

The calibration of an NDD instance corresponding to a measurement electrode configuration (MEC) may be carried out on one or more signal windows that are known not to contain evoked neural responses. In one implementation, such signal windows are “zero current” signal windows which are captured from intervals during which no stimulus is being delivered, and which have preferably been scrubbed for artefact, and may therefore be treated as comprising only noise. The calibration comprises forming estimates of parameters of a predetermined “noise model” (statistical distribution) from the samples in the one or more “zero current” signal windows. In one implementation, the noise model is Gaussian and the parameters are the mean \tilde{μ} and standard deviation \hat{σ} of the samples.

Once calibrated, an NDD instance may be applied to a signal window by counting the number \tilde{k} of outliers in the signal window, i.e. the number of samples in the signal window that depart significantly from the noise model. For a Gaussian noise model, the NDD counts the number \tilde{k} of samples that differ from the mean estimate \tilde{μ} by more than n times the standard deviation estimate \hat{σ}, where n is a small integer. The number \tilde{k} of outliers is compared to the number \hat{k} of such outliers that would be expected to occur if the signal window consisted solely of noise with mean \tilde{μ} and standard deviation \hat{σ}. The difference between \tilde{k} and \hat{k} is divided by the number of samples N in the signal window to obtain a metric r that quantifies the ratio of outliers present in a signal window relative to the expected ratio of outliers in a signal window that obeys the noise model.

It may be shown that for a Gaussian noise model, the NDD may estimate the metric r as

$$r = \frac{\tilde{k}}{N} - 2 Φ ( \frac{n}{2} ) \qquad ( 3 )$$

where Φ is the standard normal cumulative distribution function.

A negative or zero value of the metric r indicates a signal window consistent with the noise model, whereas a positive value of r indicates a departure from the noise model. Such a departure is deemed to be due to the presence of an ECAP in the signal window.

In one implementation of the NDD, n is set to 3. Smaller values of n make the NDD more sensitive, indicating a departure from noise more readily and increasing the rate of Type I errors (false positives). Conversely, high values for n necessitate large outliers before r will indicate a noise departure, increasing the rate of Type II errors (false negatives).

In one implementation of the NDD, a sigmoid function may be applied to the raw metric r to map the metric r to a quality indicator Q_{NDD} in the interval [0, 1]:

$${Q}_{N D D} = \frac{1}{1 + exp ( - γ r )} \qquad ( 4 )$$

where γ is a parameter that balances the Type I and Type II errors. The quality indicator Q_{NDD} has a natural interpretation: Q_{NDD}<0.5 corresponds to r≤0 and indicates that the signal window is most likely noise. Conversely, Q_{NDD}>0.5 indicates a departure from the noise model that is deemed to represent an ECAP. In one implementation, γ is set to 50.

In one implementation, the NDD may be applied to multiple signal windows after they have been averaged together to improve the signal-to-noise ratio. In one such implementation, the number of averaged signal windows is eight. In such implementations, the parameters of the noise model may be adjusted depending on the number of signal windows that are averaged. In the Gaussian noise model, the standard deviation \hat{σ} should be divided by the square root of the number of averaged signal windows.

### Estimating Key Patient Response Parameters

One specific aim of the APS according to the present technology is to estimate the key parameters of the patient's response to stimulus, as represented by the activation plot illustrated in FIG. 4a in idealised form. The key patient response parameters include the ECAP threshold T and the patient sensitivity S in Equation (1).

One method of forming the activation plot for a patient in a given posture involves manually increasing the stimulus intensity to the point (referred to as the perception threshold) at which the patient first perceives the stimulation, and then increasing further to a comfortable level, and then further to the discomfort threshold. The perception threshold and discomfort threshold are used to define the therapeutic window. However, patients can be unreliable and their feedback, even with honest and good intentions, is very subjective and varies with time and medication levels, as well as several other factors. Therefore, it can be difficult for patients to provide consistent and accurate responses about their sensations during programming.

A CLNS device such as the stimulator 100 is able to record neural responses (e.g. ECAPs), for which the same intensities across various postures and movements correlate to similar levels of paraesthesia and similar neural activation (i.e. the number of fibres activated), meaning that neural response intensity (e.g. ECAP amplitude) is a more reliable and consistent measure of neural activation, and therefore patient sensation, than stimulus intensity.

The methods and systems of programming a CLNS device such as the stimulator 100 according to the present technology use measurements of neural response intensity to estimate the key parameters without requiring any patient reporting of their sensations. The stimulus intensity is ramped up (increased monotonically) from zero until an evoked neural response is consistently detected, and the stimulus intensity at that point is marked as the provisional ECAP threshold. The stimulus intensity ramp continues to a perceptual marker determined from the provisional ECAP threshold, with response intensities being recorded for each stimulus. “Perceptual marker” refers to a value of stimulus that is perceptually significant for the patient. One example of a perceptual marker is the discomfort threshold described above. An activation plot model is fitted to the measured response intensities. From the fitted model, the key parameters of the patient's response may be obtained.

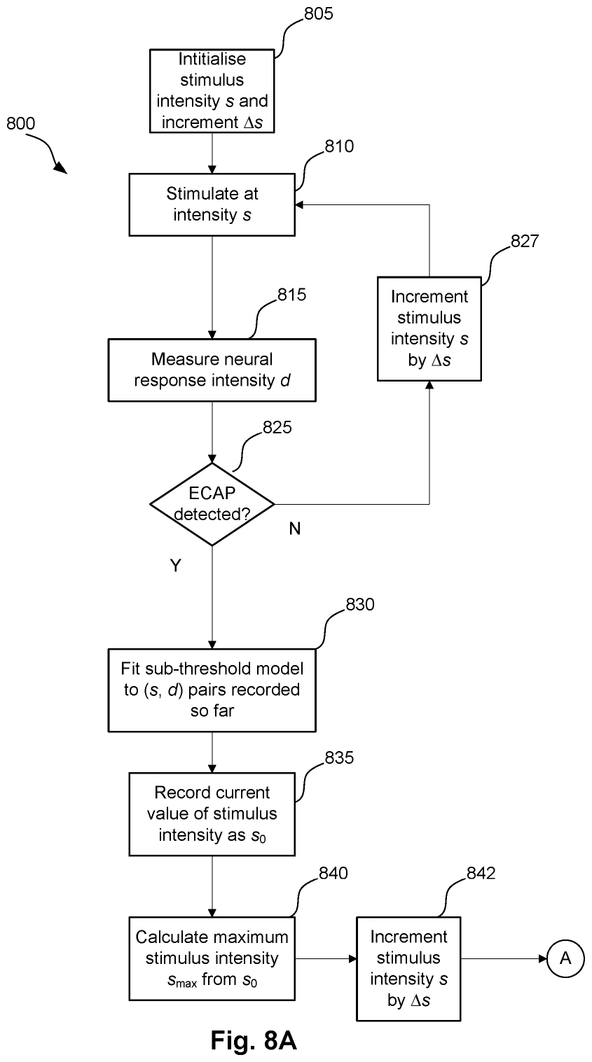

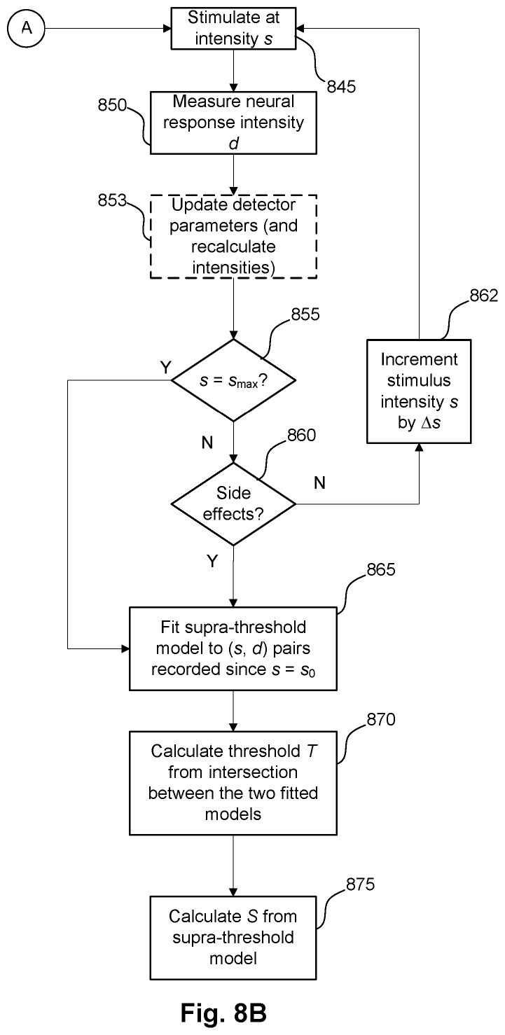

FIGS. 8A and 8B are a flow chart illustrating a method 800 of estimating key parameters of the patient's response to stimulus, according to one aspect of the present technology. The method 800 may form part of the APS as described above, and may be carried out while the patient is awake, naturally asleep, or sedated, either during implantation or post-operatively.

The method 800 starts at step 805, which initialises the stimulus intensity s to a low value, e.g. zero, and an increment As. The increment As may be initialised such that an expected ECAP threshold will be reached after a predetermined number of stimuli, or after a predetermined elapsed time (e.g. 10 seconds) at the stimulation frequency. An expected ECAP threshold for typical patients is 5 mA. Alternatively, the increment As may be initialised to a default value such as 0.1 mA. Step 810 then delivers a stimulus at the current stimulus intensity s.

Step 815 measures the intensity d of neural response in the signal window captured after the stimulus delivered at step 810. Step 815 may use an ECAP detector to measure the neural response intensity d. Step 815 records the measured response intensity d alongside the current stimulus intensity s as a pair (s, d). Depending on the form of the ECAP detector, the ECAP detector used at step 815 may return a non-zero value for d even if there is no discernible neural response in the signal window, due to the presence of artefact a and non-artefact noise n in the signal window. The measured neural response intensity d may even be negative, depending on the form of the artefact a.

The ECAP detector used at step 815 (and the later step 850, to be described below) should be of the same form as the ECAP detector 320 implemented by the electronics module 110 to be used during the eventual therapy. In one implementation, the ECAP detector used at steps 815 and 850 may be the parametric ECAP detector disclosed in the above-mentioned International Patent Publication No. WO2015/074121. The disclosed parametric ECAP detector of WO2015/074121 has two parameters: correlation delay and length. The ECAP detector at step 815 may have been configured with default detector parameters based on typical ECAP signal conduction velocity and the known distance between the stimulation electrode(s) and the measurement electrodes.

Step 825 then checks whether a neural response (ECAP) has been detected in the current signal window, or multiple recently processed signal windows. In one implementation, step 825 uses the NDD described above to detect the presence of an ECAP. In one such implementation, step 825 detects an ECAP if the NDD returns a quality indicator Q_{NDD} of 50% (0.5), i.e. if the NDD detects an ECAP in at least 50% of the signal windows processed at the current stimulus intensity. In another implementation, step 825 uses a basis-element-signal-separation-(BESS)-based ECAP detector. A BESS-based ECAP detector estimates the underlying component signals (e.g. ECAP and artefact) of the signal window given only the signal window, and without knowledge of the exact underlying ECAP and artefact component signals. Various BESS-based ECAP detectors are described in the above-mentioned International Publication No. WO2020/124135. If the BESS-based ECAP detector returns a positive value above some predetermined noise threshold for the amplitude d of the ECAP component in the signal window, an ECAP has been detected. In one such implementation, step 825 detects an ECAP if the BESS-based ECAP detector detects an ECAP in at least 50% of the signal windows processed at the current stimulus intensity.

In other implementations of step 825, methods of determining a likelihood that a captured signal window contains an ECAP are disclosed in International Patent Publication No. PCT/AU2022/051583, the contents of which are herein incorporated by reference. The likelihood returned by such methods may be compared with a threshold, such as 0.5, to determine whether the captured signal window contains an ECAP.

If step 825 does not detect a neural response (“N”), the method 800 at step 827 increments the stimulus intensity by the predetermined increment As and returns to step 810. If step 825 does detect a neural response (“Y”), step 830 fits a model to the ensemble of pairs (s, d) recorded at the multiple iterations of step 815 since the start of the method 800 at s=0. The model fitted at step 830 is referred to as the sub-threshold model. The sub-threshold model may be a straight line, in which case step 830 may use linear regression to fit the model. Step 835 then records the current stimulus intensity s as the provisional ECAP threshold _{S0}.

Note that the APS may omit the incrementing in certain iterations of step 827, so that multiple consecutive stimuli are delivered at the same intensity before the next increment takes place. This effectively decreases the rate of increase of stimulus intensity s per unit time. In such implementations the increment Δ_{S} may be predetermined such that an expected ECAP threshold will be reached after a predetermined elapsed time (e.g. 10 seconds).

Step 840 then calculates a value _{Smax} for the perceptual marker that will mark the upper limit of the stimulus intensity ramp from the provisional ECAP threshold _{S0}. In one implementation, step 840 uses a linear prediction model to obtain the perceptual marker from the provisional ECAP threshold _{S0}:

$${s}_{max} = m \cdot {s}_{0} \qquad ( 5 )$$

where m is a correlation parameter that may be derived from historical patient data relating patients' ECAP thresholds with their respective perceptual markers. In some implementations in which the perceptual marker is a discomfort threshold, m takes a value between 1.0 and 2.0. In another implementation, m takes a value between 1.1 and 1.6. In some such implementations, m takes a value between 1.25 and 1.54. Step 840 may use a different correlation parameter m depending on whether the patient is awake, naturally asleep, or sedated, as a perceptual marker is likely to vary depending on the sleep state of the patient. In the case of a sedated patient, the perceptual marker may be a motor threshold at which motor fibres are activated by the stimulus.

Step 842 then increments the stimulus intensity by the predetermined increment Δ_{S}.

The method 800 then proceeds to step 845, which delivers a stimulus at the current stimulus intensity s. Step 850 then measures the intensity d of a neural response in the signal window captured after the delivered stimulus at step 845, in similar fashion to, and using the same ECAP detector as, step 815. Step 850 records the measured response intensity d alongside the current stimulus intensity s as a pair (s, d).

In the implementation in which the ECAP detector used at steps 815 and 850 is the parametric ECAP detector disclosed in the above-mentioned International Patent Publication No. WO2015/074121, the APS may, in an optional step 853, dynamically update the parameters (e.g. length and correlation delay) of the parametric ECAP detector on an average signal window obtained by averaging a predetermined number of recent signal windows, for example 10. The parametric ECAP detector is applied to measure the ECAP amplitude in the average signal window for every feasible value of correlation delay and length to form a correlation matrix. The values of correlation delay and length that maximise the measured ECAP amplitude within the correlation matrix are chosen as optimal. The APS may also, at step 853, retrospectively re-measure the response intensities d for all signal windows captured since the start of the method 800 using the updated ECAP detector parameters.

Step 855 then tests whether the stimulus intensity s has reached the perceptual marker _{Smax}. If not (“N”), step 860 then tests whether any adverse side effects of the increasing stimulus intensity have been experienced. In one implementation, an indicator of an adverse side effect is the appearance of a “late” or “slow” response after the ECAP in the signal window. The late response, which may be indicative of motor fibre activation, may be detected as described in International Patent Publication no. WO2015/070281, the contents of which are herein incorporated by reference. Another indicator of an adverse side effect is the saturation (flattening out) of neural response intensity with continuing increments in the stimulus intensity.

If no side effects are detected (“N”) at step 860, the method 800 increments the stimulus intensity s by the increment Δ_{S} at step 862. As with step 827, the APS may omit the incrementing in certain iterations of step 845, so that multiple consecutive stimuli are delivered at the same intensity before the next increment takes place. The method 800 then returns to step 845. If however either the stimulus intensity s has reached the perceptual marker _{Smax} (“Y” at step 855), or at least one adverse side effect is detected from the increasing stimulus intensity (“Y” at step 860), no further stimuli are delivered, and the method 800 proceeds to step 865. Step 865 fits a model to the ensemble of pairs (s, d) recorded at the multiple iterations of step 850 since step 840 at which stimulus intensity s was equal to the provisional ECAP threshold _{S0}. The model fitted at step 865 is referred to as the supra-threshold model. The supra-threshold model may be a straight line, in which case step 865 may use linear regression to fit the model.

In implementations in which step 853 re-measures the neural response intensities, the sub-threshold model fitted to the sub-threshold pairs (s, d) at step 830 may be re-fitted at step 865 using the re-measured intensities.

Step 870 then calculates the value of stimulus intensity at which the sub-threshold model fitted at step 830 or re-fitted at step 865 and the supra-threshold model fitted at step 865 intersect. The calculated value of stimulus intensity is the ECAP threshold T.

Finally, step 875 computes the patient sensitivity S from the supra-threshold model. In the implementation in which the supra-threshold model is linear, the sensitivity S is the slope of the fitted line.

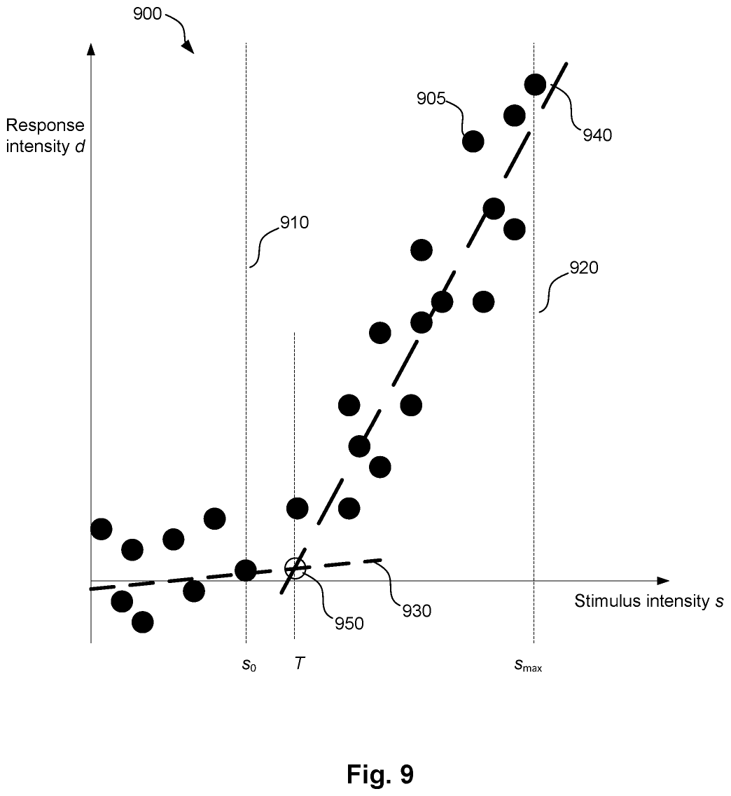

FIG. 9 is a graph 900 illustrating the operation of the method 800. Each point (e.g. the point 905) represents a recorded (s, d) pair on axes representing stimulus intensity s and response intensity d. The vertical dashed line 910 indicates the provisional ECAP threshold _{S0}, at which an ECAP was first detected. The vertical dashed line 920 indicates the perceptual marker _{Smax}, derived from the provisional ECAP threshold _{S0}. The dashed line 930 represents the sub-threshold model as a linear model of the sub-threshold pairs. The dashed line 940 represents the supra-threshold model as a linear model of the supra-threshold pairs. The point 950 is the intersection between the sub-threshold model 930 and the supra-threshold model 940. The s-coordinate of the intersection point 950 is the ECAP threshold T. The ECAP threshold T in FIG. 9 is slightly different from the provisional ECAP threshold _{S0}. The slope of the supra-threshold model 940 is the sensitivity S.

An alternative supra-threshold model to be fit at step 865 is the logistic growth curve (LGC) model (also referred to as a sigmoid function). In one implementation, the LGC model is a four-parameter function:

$$d ( s ) = A + \frac{K - A}{1 + exp ( - B ( s - M ) )} \qquad ( 6 )$$

where the four parameters are:
A, the minimum value (the detected ECAP amplitude in the absence of stimulation)
K, the maximum value (the detected ECAP amplitude at which saturation occurs, i.e. increases in stimulus intensity do not increase the detected ECAP amplitude)
M, the current amplitude at the midpoint between A and K
B, the steepness of the LGC, which is proportional to the gradient at the midpoint between A and K.

In other implementations, fewer parameters may be used for the LGC model, for example an LGC model in which the minimum value A is identically zero.

To fit the LGC, the parameters A, K, M, and B may be initialised to sensible starting points A₀, K₀, M₀, and B₀. In one implementation, these values may be set to:
A₀: the mean of the response intensities obtained from the lowest few stimulus intensities.
K₀: the mean of the response intensities obtained from the highest few stimulus intensities.
M₀: the stimulus current amplitude at the midpoint between A and K.
B₀: may be calculated from the gradient m at the midpoint, obtained from local linear regression of pairs acquired near the midpoint, as B₀ =m*4/(K₀−A₀).

An optimisation algorithm such as Trust Region Reflective (TRF) may then be used to optimise the four parameters A, K, M, and B from their starting points A₀, K₀, M₀, and B₀.

The fitted LGC may be used to estimate the patient sensitivity, as in step 875 of the method 800. In one implementation, the patient sensitivity S is the slope of the fitted LGC at its midpoint M, which may be computed from the steepness B as follows:

$$S = \frac{B}{4} ( K - A ) \qquad ( 7 )$$

The point of intersection between a linear sub-threshold model d(s)=m*s+b and an LGC supra-threshold model as in Equation (6) may be found by solving the following equation numerically for s:

$$m s + b = A + \frac{K - A}{1 + {e}^{- B ( s - M )}} \qquad ( 8 )$$

Once the key patient response parameters have been estimated, e.g. by the method 800, they may be used by the APS to determine clinical settings (therapy parameters) for the CLNS system 300.

In one implementation, the measured sensitivity S (possibly adjusted for effective stimulus frequency) may be used to set the gain K of the gain element 336 for the corresponding SEC. International Patent Publication no. WO2016090436, the contents of which are incorporated herein by reference, describes how the controller gain K may be set based on the measured sensitivity S. In another implementation, the controller gain K may be computed as

$$K = \frac{cos ω - 1 + \sqrt{{cos}^{2} ω - 4 cos ω + 3}}{S} \qquad ( 9 )$$

where

$$ω = \frac{2 π {f}_{c}}{{f}_{s}} ,$$

ƒ_{c} is a loop cutoff frequency, and ƒₛ is the stimulus frequency. In one implementation, the loop cutoff frequency is set to 3 Hz to balance the attenuation of noise with the attenuation of postural disturbances such as heartbeat.

In another implementation, the initial target ECAP amplitude may be set based on the measured ECAP threshold T and the measured sensitivity S.

In another implementation according to the present technology, a variant of the method 800, instead of estimating key response parameters at steps 865 to 875, outputs a binary-valued indicator at step 865 depending on whether the perceptual marker was reached (i.e. the “Y” outcome of step 855) or a side effect occurred (i.e. the “Y” outcome of step 860). If the perceptual marker was reached, the indicator indicates that the perceptual marker was reached without side effects. If a side effect occurred, the indicator indicates that side effects occurred before the perceptual marker was reached. The variant of the method 800 then either halts, or returns to step 862 to continue the ramp to another, higher perceptual marker and return a similar indicator for the higher perceptual marker.

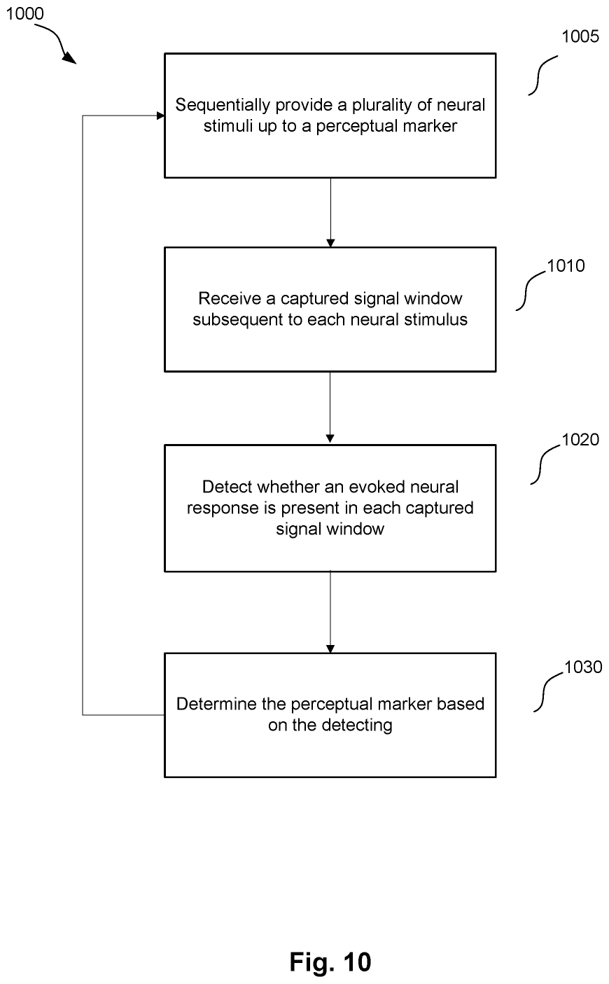

FIG. 10 is a flow chart illustrating a method 1000 according to one aspect of the present technology. The method 1000 may form part of the APS as described above, and may be carried out while the patient is awake, naturally asleep, or sedated, either during implantation or post-operatively.

The method 1000 starts at step 1005, which sequentially delivers a plurality of neural stimuli to neural tissue of a patient according to a ramp of stimulus intensity parameter values up to a perceptual marker. Step 1010 receives a captured signal window subsequent to each delivered neural stimulus. Step 1020 detects whether an evoked neural response is present in each captured signal window. Step 1030 determines the perceptual marker based on the detecting. The method 1000 then cycles back to step 1005 to continue the ramp of stimulus intensity parameters up to the perceptual marker.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

### Label list

| | |
|---|---|
| stimulator | 100 |
| Patient | 108 |
| electronics module | 110 |
| Battery | 112 |
| telemetry module | 114 |
| controller | 116 |
| memory | 118 |
| clinical data | 120 |
| clinical settings | 121 |
| transcutaneous | |
| control programs | 122 |
| pulse generator | 124 |
| electrode selection module | 126 |
| measurement circuitry | 128 |
| system ground | 130 |
| electrode array | 150 |
| current pulse | 160 |
| ECAP | 170 |
| nerve | 180 |
| communications channel | 190 |
| external device | 192 |
| CLNS system | 300 |
| clinical settings controller | 302 |
| target ECAP controller | 304 |
| Box | 308 |
| Box | 309 |
| controller | 310 |
| box | 311 |
| stimulator | 312 |
| element | 313 |
| measurement circuitry | 318 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| target ECAP amplitude | 520 |
| ECAP | 600 |
| neural stimulation system | 700 |
| neuromodulation device | 710 |
| remote controller | 720 |
| clinical settings transceiver | 730 |
| clinical interface | 740 |
| charger | 750 |
| method | 800 |
| step | 805 |
| step | 810 |
| step | 815 |
| step | 825 |
| step | 827 |
| step | 830 |
| step | 835 |
| step | 840 |
| step | 842 |
| step | 845 |
| step | 850 |
| step | 853 |
| step | 855 |
| step | 860 |
| step | 863 |
| step | 865 |
| step | 870 |
| step | 875 |
| graph | 900 |
| point | 905 |
| vertical dashed line | 910 |
| vertical dashed line | 920 |
| dashed line | 930 |
| supra - threshold model | 940 |
| intersection point | 950 |
| method | 1000 |
| step | 1005 |
| step | 1010 |
| step | 1020 |
| step | 1030 |

1. A neural stimulation system comprising:
a neuromodulation device for delivering neural stimuli, the neuromodulation device comprising:
a stimulus source configured to provide neural stimuli to be delivered via one or more stimulus electrodes to neural tissue of a patient, the neural stimuli being configured to evoke neural responses in the neural tissue;
measurement circuitry configured to capture signal windows from signals sensed on the neural tissue via one or more sense electrodes; and
a control unit configured to control the stimulus source to provide each neural stimulus according to a stimulus intensity parameter; and
a processor configured to:
instruct the control unit to control the stimulus source to sequentially provide a plurality of neural stimuli according to a ramp of stimulus intensity parameter values up to a perceptual marker;
receive a captured signal window subsequent to each neural stimulus;
detect whether an evoked neural response is present in each captured signal window; and
determine the perceptual marker based on the detecting.

2. The neural stimulation system of claim 1, wherein the processor is further configured to:
determine a provisional neural response threshold based on the detecting; and
determine the perceptual marker from the provisional neural response threshold.

3. The neural stimulation system of claim 2, wherein the processor is configured to determine the perceptual marker from the provisional neural response threshold using a linear prediction model.

4. The neural stimulation system of claim 2, wherein the processor is configured to determine the provisional neural response threshold as the lowest stimulus intensity parameter at which the detecting detects evoked neural responses in the captured signal windows.

5. The neural stimulation system of claim 1, wherein the processor is configured to detect whether an evoked neural response is present in a captured signal window by detecting a statistically unusual difference from noise expected to be present in the signal window.

6. The neural stimulation system of claim 5, wherein the processor is configured to detect whether an evoked neural response is present in a captured signal window by first removing artefact from the signal window.

7. The neural stimulation system of claim 2, wherein the processor is further configured to measure an intensity of an evoked neural response in each captured signal window, thereby forming a stimulus intensity parameter-response intensity pair for each neural stimulus.

8. The neural stimulation system of claim 7, wherein the processor is further configured to fit a sub-threshold model to stimulus intensity parameter-response intensity pairs with stimulus intensity values up to the provisional neural response threshold.

9. The neural stimulation system of claim 8, wherein the processor is further configured to fit a supra-threshold model to stimulus intensity parameter-response intensity pairs with stimulus intensity values above the provisional neural response threshold.

10. The neural stimulation system of claim 9, wherein the processor is further configured to determine a key parameter of the response of the neural tissue to neural stimuli from the supra-threshold model.

11. The neural stimulation system of claim 10, wherein the key parameter is a neural response threshold, and the processor is configured to determine the neural response threshold from a point of intersection of the sub-threshold model and the supra-threshold model.

12. The neural stimulation system of claim 10, wherein the key parameter is a sensitivity, and the processor is configured to determine the key parameter by measuring the sensitivity from the supra-threshold model.

13. The neural stimulation system of claim 7, wherein the processor is further configured to measure the intensity of an evoked neural response in each captured signal window using a parametric evoked compound action potential (ECAP) detector comprising one or more parameters.

14. The neural stimulation system of claim 13, wherein the processor is further configured to update one or more parameters of the parametric ECAP detector during the ramp of stimulus intensity parameter values.

15. The neural stimulation system of claim 14, wherein the processor is further configured to re-measure the measured intensities of the evoked neural responses using the updated parameters of the parametric ECAP detector.

16. The neural stimulation system of claim 1, wherein the processor is further configured to halt the ramp of stimulus intensity parameter values upon detecting an adverse side effect of the neural stimuli.

17. The neural stimulation system of claim 1, wherein the processor is part of the neuromodulation device.

18. The neural stimulation system of claim 1, further comprising an external computing device in communication with the neuromodulation device.

19. The neural stimulation system of claim 18, wherein the processor is part of the external computing device.

20. An automated method of delivering neural stimuli to neural tissue of a patient, the method comprising:
sequentially delivering a plurality of neural stimuli to the neural tissue according to a ramp of stimulus intensity parameter values up to a perceptual marker;
receiving a captured signal window subsequent to each delivered neural stimulus;
detecting whether an evoked neural response is present in each captured signal window; and
determining the perceptual marker based on the detecting.

21. The method of claim 20, further comprising:
determining a provisional neural response threshold based on the detecting; and
determining the perceptual marker from the provisional neural response threshold.

22. The method of claim 21, wherein the determining of the perceptual marker from the provisional neural response threshold uses a linear prediction model.

23. The method of claim 21, wherein the determining the provisional neural response threshold comprises determining the lowest stimulus intensity parameter at which the detecting detects evoked neural responses in the captured signal windows.

24. The method of claim 20, wherein detecting whether an evoked neural response is present in a captured signal window comprises detecting a statistically unusual difference from noise expected to be present in the signal window.

25. The method of claim 24, wherein the detecting whether an evoked neural response is present in a captured signal window comprises first removing artefact from the signal window.

26. The method of claim 21, further comprising measuring an intensity of an evoked neural response in each captured signal window, thereby forming a stimulus intensity parameter-response intensity pair for each neural stimulus.

27. The method of claim 26, further comprising fitting a sub-threshold model to stimulus intensity parameter-response intensity pairs with stimulus intensity values up to the provisional neural response threshold.

28. The method of claim 27, further comprising fitting a supra-threshold model to stimulus intensity parameter-response intensity pairs with stimulus intensity values above the provisional neural response threshold.

29. The method of claim 28, further comprising determining a key parameter of the response of the neural tissue to neural stimuli from the supra-threshold model.

30. The method of claim 29, wherein the key parameter is a neural response threshold, and the determining the key parameter comprises determining the neural response threshold from a point of intersection of the sub-threshold model and the supra-threshold model.

31. The method of claim 29, wherein the key parameter is a sensitivity, and the determining the key parameter comprises measuring the sensitivity from the supra-threshold model.

32. The method of claim 26, further comprising measuring the intensity of an evoked neural response in each captured signal window using a parametric ECAP detector comprising one or more parameters.

33. The method of claim 32, further comprising updating one or more parameters of the parametric ECAP detector during the ramp of stimulus intensity parameter values.

34. The method of claim 33, further comprising re-measuring the measured intensities of the evoked neural responses using the updated parameters of the parametric ECAP detector.

35. The method of claim 20, further comprising halting the ramp of stimulus intensity parameter values upon detecting an adverse side effect of the neural stimuli.

36. A neural stimulation system comprising:
a neuromodulation device for delivering neural stimuli, the neuromodulation device comprising:
a stimulus source configured to provide neural stimuli to be delivered via one or more stimulus electrodes to neural tissue of a patient, the neural stimuli being configured to evoke neural responses in the neural tissue;
measurement circuitry configured to capture signal windows from signals sensed on the neural tissue via one or more sense electrodes; and
a control unit configured to control the stimulus source to provide each neural stimulus according to a stimulus intensity parameter; and
a processor configured to:
instruct the control unit to control the stimulus source to provide one or more neural stimuli according to a stimulus intensity parameter value;
detect whether an evoked neural response is present in each captured signal window;
increment the stimulus intensity parameter value;
repeat the instructing, detecting, and incrementing until the stimulus intensity parameter value is equal to a perceptual marker; and
determine the perceptual marker based on the detecting.

37. The neural stimulation system of claim 36, wherein the control unit is further configured to measure an intensity of an evoked neural response in each captured signal window subsequent to a neural stimulus, thereby forming a stimulus intensity parameter value-response intensity pair for each neural stimulus.

38. An automated method of delivering neural stimuli to neural tissue of a patient, the method comprising:
delivering one or more neural stimuli to the neural tissue according to a stimulus intensity parameter value;
detecting whether an evoked neural response is present in each captured signal window;
incrementing the stimulus intensity parameter value;
repeating the delivering, detecting, and incrementing until the stimulus intensity parameter value is equal to a perceptual marker; and
determining the perceptual marker based on the detecting.

39. The method of claim 38, further comprising measuring an intensity of an evoked neural response in each captured signal window subsequent to a neural stimulus, thereby forming a stimulus intensity parameter value-response intensity pair for each neural stimulus.
